(19) United States
(12) Patent Application Publication
(10) Pub. No.: US 2017/0142501 A1
(43) Pub. Date: May 18, 2017

(54) INTERNET OF SHOES

(71) Applicant: QUALCOMM Incorporated, San Diego, CA (US)

(72) Inventor: Bjorn Markus Jakobsson, Portola Valley, CA (US)

(21) Appl. No.: 14/942,550

(22) Filed: Nov. 16, 2015

(51) Int. Cl. H04Q 9/00; A61B 5/0205; H02J 7/32; A61B 5/00; G08B 21/04; G01C 21/36

(52) U.S. Cl. CPC H04Q 9/00; G08B 21/0446; G01C 21/3652; H02J 7/32; A61B 5/6807; A61B 5/02055; A61B 5/486; H04Q 2209/40; A61B 2562/0223; A61B 2560/0214; A61B 2560/0209

(57) ABSTRACT

Aspects of the disclosure are related to a connected shoe apparatus, comprising: a processor; a memory coupled to the processor; a radio; an antenna; and a magnetometer, wherein the connected shoe apparatus is wearable as a shoe by a user, and wherein the processor is to: determine a direction the connected shoe apparatus is facing with the magnetometer, and transmit information associated with the direction to a second device via the radio and the antenna.

### FIELD

The subject matter disclosed herein relates to wearable electronic devices, and more particularly to a connected shoe.

### BACKGROUNDS

While a smartphone has many applications, it also has its restrictions. For example, a magnetometer-equipped smartphone can provide useful direction information based on magnetometer readings only when a user intentionally holds it in a particular way. Further, using a smartphone for pedestrian navigation requires the user to frequently check the phone. It is inconvenient and distracting, and exposes the user as not familiar with the geographical region. It is also difficult to use the navigation function while the user is talking on the phone at the same time. Moreover, smartphone-based gait detection has poor results due to phone-associated limitations (e.g., some characteristics are removed by body damping; some are fuzzed by change of shoes; a smartphone generally has only one accelerometer in one location, etc.). Still further, a smartphone is not a suitable wearable identity manager, and not all users are willing to wear a smartwatch or bracelet.

### SUMMARY

One aspect of the disclosure is related to a connected shoe apparatus, comprising: a processor; a memory coupled to the processor; a radio; an antenna; and a magnetometer, wherein the connected shoe apparatus is wearable as a shoe by a user, and wherein the processor is to: determine a direction the connected shoe apparatus is facing with the magnetometer, and transmit information associated with the direction to a second device via the radio and the antenna.

Another aspect of the disclosure is related to a method, comprising: determining a direction a connected shoe apparatus is facing with a magnetometer; and transmitting information associated with the direction to a second device via a radio and an antenna, wherein the connected shoe apparatus is wearable as a shoe by a user.

Yet another aspect of the disclosure is related to a connected shoe apparatus, comprising: means for determining a direction the connected shoe apparatus is facing; and means for transmitting information associated with the direction to a second device, wherein the connected shoe apparatus is wearable as a shoe by a user.

Still another aspect of the disclosure is related to a non-transitory computer-readable medium comprising code which, when executed by a processor of a connected shoe wearable by a user, causes the processor to perform a method comprising: determining a direction the connected shoe is facing with a magnetometer; and transmitting information associated with the direction to a second device via a radio and an antenna.

Still another aspect of the disclosure is related to an article of footwear, comprising: a processor; a memory coupled to the processor; a radio; an antenna; and a magnetometer, wherein the article of footwear is wearable as a footwear by a user, and wherein the processor is to: determine a direction the article of footwear is facing with a magnetometer, and transmit information associated with the direction to a second device via the radio and the antenna.

### BRIEF DESCRIPTION OF THE DRAWINGS

FIG. 1 is diagram illustrating an example device with which embodiments of the disclosure may be practiced.

FIG. 2 is a diagram illustrating various components of an example connected shoe.

FIG. 3 is an example illustration of shoe-based tactile notification.

FIG. 4 is a flowchart illustrating an example method for using a connected shoe to determine a direction.

FIG. 5 is a flowchart illustrating an example method for tactile notification-based navigation using a connected shoe.

FIG. 6 a flowchart illustrating an example method for detecting gait biometrics using a connected shoe.

FIG. 7 is a flowchart illustrating an example method for using a connected shoe as a wearable identity manager.

FIG. 8 is a flowchart illustrating an example method for using a connected shoe as a health monitor.

### DETAILED DESCRIPTION

Embodiments of the disclosure are directed to a connected shoe (article of footwear). The connected shoe may comprise a battery, radio(s) and antenna(s) for wireless communication with one or more other connected devices, a memory, a processor or a simpler controller, and any of a number of other electronic components, such as a magnetometer for direction detection, one or more vibration motors that may provide tactile notification to the user for navigation or other purposes, piezoelectric components that may harvest energy from the user's walking and charge the battery, one or more pressure sensor pads and/or a motion sensor that may be used for gait detection, and/or one or more physiological sensors such as a pulse sensor, a blood pressure sensor, a body temperature sensor, etc. The wireless communication may be based on such low energy wireless connection protocols as Bluetooth low energy (BLE).

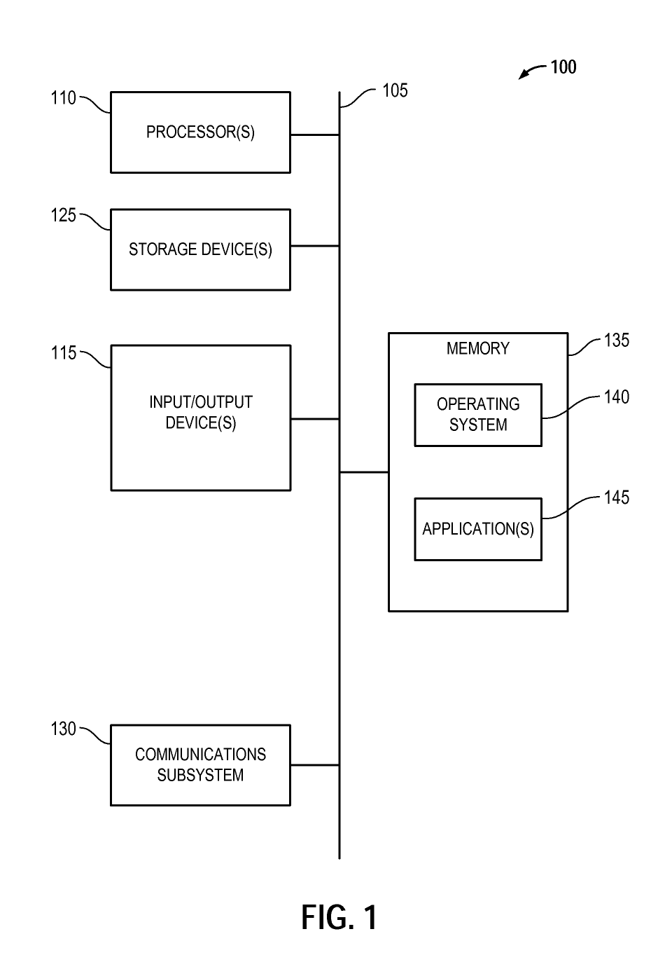

Referring to FIG. 1, an example device 100 adapted for connecting with wearable electronic devices is shown. The device 100 is shown comprising hardware elements that can be electrically coupled via a bus 105 (or may otherwise be in communication, as appropriate). The hardware elements may include one or more processors 110, including without limitation one or more general-purpose processors and/or one or more special-purpose processors (such as digital signal processing chips, graphics acceleration processors, and/or the like); one or more input/output devices 115, a mouse, a keyboard, a speaker, a printer, and/or the like.

The device 100 may further include (and/or be in communication with) one or more non-transitory storage devices 125, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including without limitation, various file systems, database structures, and/or the like.

The device 100 might also include a communications subsystem 130, which can include without limitation a modem, a network card (wireless or wired), an infrared communication device, a wireless communication device and/or chipset (such as a Bluetooth device, an 802.11 device, a Wi-Fi device, a WiMAX device, cellular communication facilities, etc.), and/or the like. The communications subsystem 130 may permit data to be exchanged with a network, other computer systems/devices, and/or any other devices described herein. In many embodiments, the device 100 will further comprise a working memory 135, which can include a RAM or ROM device, as described above.

The device 100 also can comprise software elements, shown as being currently located within the working memory 135, including an operating system 140, device drivers, executable libraries, and/or other code, such as one or more application programs 145, which may comprise or may be designed to implement methods, and/or configure systems, provided by other embodiments, as described herein. Merely by way of example, one or more procedures described with respect to the method(s) discussed below might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be stored on a non-transitory computer-readable storage medium, such as the storage device(s) 125 described above. In some cases, the storage medium might be incorporated within a computer device, such as the device 100. In other embodiments, the storage medium might be separate from a computer device (e.g., a removable medium, such as a compact disc), and/or provided in an installation package, such that the storage medium can be used to program, configure, and/or adapt a general purpose computer with the instructions/code stored thereon. These instructions might take the form of executable code, which is executable by the device 100 and/or might take the form of source and/or installable code, which, upon compilation and/or installation on the device 100 (e.g., using any of a variety of generally available compilers, installation programs, compression/decompression utilities, etc.), then takes the form of executable code.

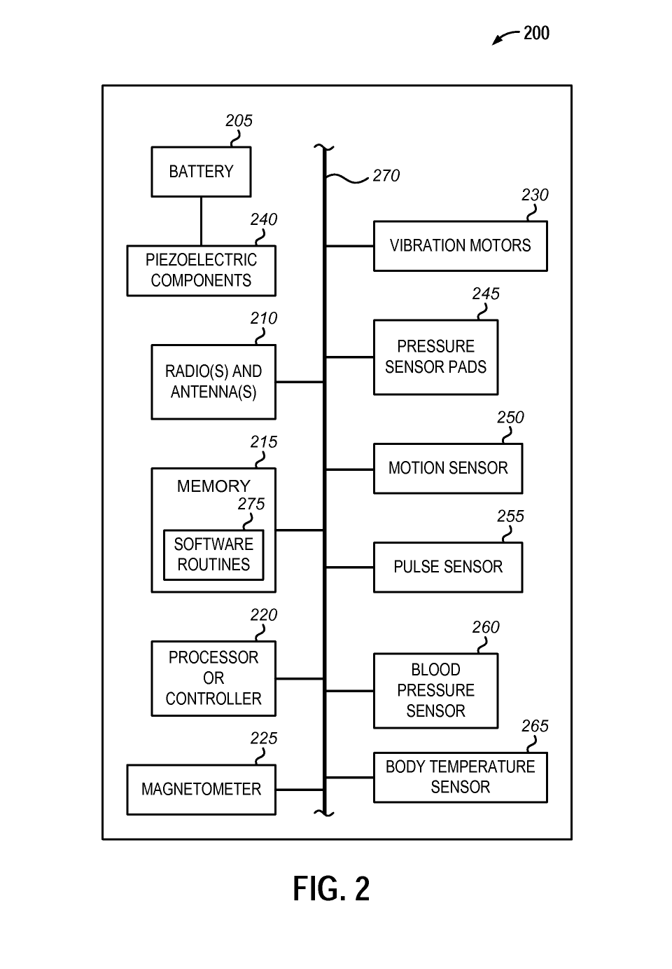

Referring to FIG. 2, a diagram illustrating various components of an example connected shoe 200. The connected shoe 200 may comprise without limitation a battery 205, radio(s) and antenna(s) 210, a memory 215, a processor or controller 220, a magnetometer 225, one or more vibration motors 230, one or more piezoelectric components 240, one or more pressure sensor pads 245, a motion sensor 250, a pulse sensor 255, a blood pressure sensor 260, and a body temperature sensor 265. The memory 215, the processor/controller 220, the radio(s) and antenna(s) 210, the vibration motors 230, and the various sensors are interconnected using a bus 270 or other suitable connections. As non-limiting examples, the radio(s) and antenna(s) 210 may support such protocols as Bluetooth, BLE, IEEE 802.11 wireless local area network (WLAN), cell phone networks (such as GSM, CDMA, UMTS, CDMA2000, LTE, LTE Advanced, etc.). Software routines 275, executable code, etc. may be stored in the memory 215 to enable and support the various functions of the connected shoe 200. It should be noted that not all the above-listed components are required for any particular embodiment of the disclosure. It should further be appreciated that within a pair of shoes, the various electronic components may be deployed within one shoe, may be distributed between the two shoes, as appropriate, or may be duplicated in both shoes. When the electronic components are distributed between the two shoes or duplicated in both shoes, the electronic components within the two shoes may communicate wirelessly, as appropriate and/or necessary.

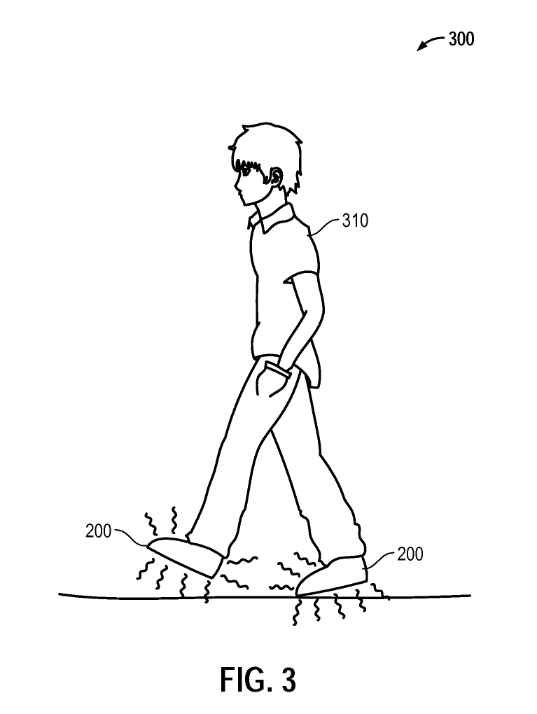

Referring to FIG. 3, an example illustration 300 of shoe-based tactile notification is shown. The connected shoes 200 may activate one or more vibration motors 230 to provide tactile notification to a user 310 for navigation or other suitable purposes. In one embodiment, each shoe 200 is equipped with four vibration motors 230—front, rear, left, and right. The left vibration motors 230 may vibrate when, for example, it is time to make a left turn. In another embodiment, the shoes 200 may activate one or more vibration motors 230 to prompt the user 310 to increase the step frequency in order to reach preset exercise goals. In yet another embodiment, each connected shoe 200 within a pair of shoes may comprise one vibration motor 230. The vibration motor 230 in the left shoe may vibrate when, for example, it is time to make a left turn.

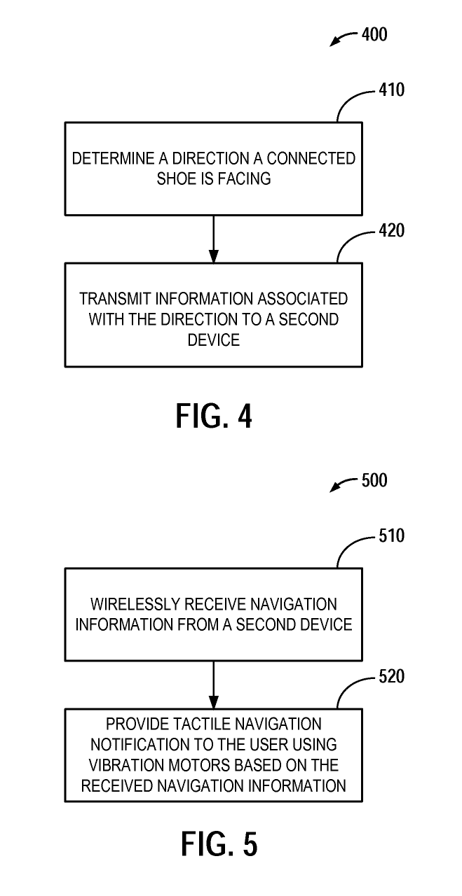

Referring to FIG. 4, a flowchart illustrating an example method 400 for using a connected shoe to determine a direction is shown. At block 410, a direction a connected shoe 200 is facing may be determined using the magnetometer. At block 420, information associated with the direction is wirelessly transmitted to a second device, such as the device 100 of FIG. 1, from the connected shoe 200.

Referring to FIG. 5, a flowchart illustrating an example method 500 for tactile notification-based navigation using a connected shoe is shown. At block 510, the connected shoe may wirelessly receive navigation information from a second device, such as the device 100 of FIG. 1. The navigation information may contain turn directions. At block 520, tactile notification may be provided to the user based on the received navigation information. For example, each shoe 200 may be equipped with four vibration motors 230—front, rear, left, and right. Therefore, as a non-limiting example, the connected shoe 200 may activate the left vibration motor 230 at an intersection when the navigation information indicates it is time to make a left turn, may activate the right vibration motor 230 when the navigation information indicates it is time to make a right turn, may activate the front vibration motor 230 when the navigation information indicates that the user should continue forward, and may activate the rear vibration motor 230 when the navigation information indicates that the user should turn around and walk back. In another embodiment, each shoe 200 within a pair of shoes may be equipped with one vibration motor 230. Therefore, the vibration motor 230 in the left shoe may be activated when the navigation information indicates it is time to make a left turn; the vibration motor 230 in the right shoe may be activated when the navigation information indicates it is time to make a right turn; and the vibration motors 230 in both shoes 200 may be activated to implement a first vibration pattern when the navigation information indicates that the user should continue forward and may be activated to implement a second vibration pattern when the navigation information indicates that the user should turn around and walk back. The second device, such as the device 100 of FIG. 1, may be preconfigured with the navigation destination so that it can generate the navigation information. As non-limiting examples, the navigation destination may be manually entered at the device 100 or entered by using the device 100 to scan a Quick Response (QR) or another suitable code which contains or references information about the navigation destination.

If the connected shoe 200 is equipped with suitably positioned piezoelectric components 240 that harvest energy from the user's walking and recharge the battery 205, the connected shoe 200 may have sufficient energy to help off-load some power-intensive operations including radio operations of the second device, such as the device 100 of FIG. 1, so that the second device may have more battery power for other operations. For example, the connected shoe 200 may maintain a wireless connection with the second device over BLE, which draws minimal power. When the second device needs to transmit a signal over a WLAN or cell network, it may transmit the signal to the connected shoe 200 over BLE, and thereafter the connected shoe 200 may retransmit the signal over the correct network, be it the WLAN or cell network. In the other direction, the connected shoe 200 may receive WLAN and/or cell network signals intended for the second device, and may retransmit the signal to the second device over BLE. Therefore, the second device may save energy without reduction in actual wireless communications. In some embodiments, the connected shoe 200 may help off-load complex data processing operations of the second device by receiving the data to be processed from the second device over BLE, processing the data at the connected shoe 200, and transmitting the processed data back to the second device.

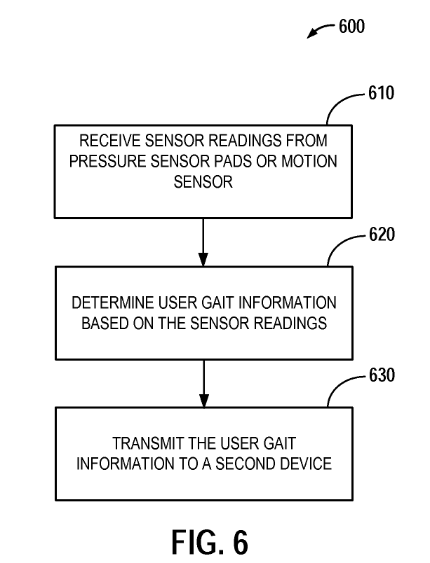

Referring to FIG. 6, a flowchart illustrating an example method 600 for detecting user gait biometrics using a connected shoe is shown. At block 610, sensor readings may be received from the pressure sensor pads 245 or the motion sensor 250. At block 620, user gait information may be determined based on the sensor readings. At block 630, the user gait information may be wirelessly transmitted to a second device, such as the device 100 of FIG. 1, from the connected shoe 200. In one embodiment, the connected shoe 200 may comprise four pressure sensor pads 245—front, rear, left, and right. Such gait-based biometric identification/authentication is difficult to defeat even for family members or close friends of the intended user because of different shoe sizes. Of course, in some embodiments, the sensor readings may be transmitted directly to the second device, wherein the user gait information may be determined at the second device.

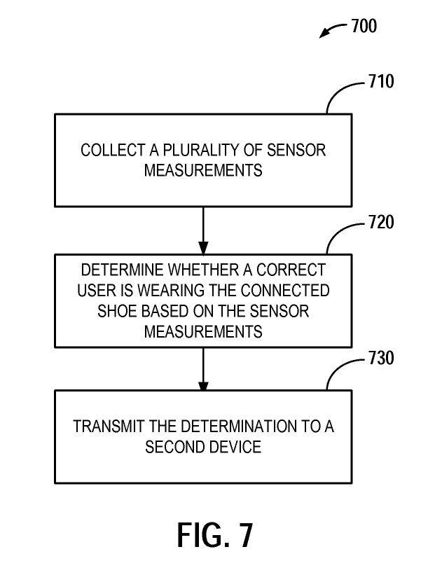

Referring to FIG. 7, a flowchart illustrating an example method 700 for using a connected shoe as a wearable identity manager is shown. At block 710, a plurality of sensor measurements may be collected by a connected shoe 200. These may include, e.g., pressure levels detected using pressure sensor pads 245, body heat levels detected using the body temperature sensor 265, or user pulse characteristics detected using the pulse sensor 255, etc. At block 720, it may be determined whether a correct user is wearing the connected shoe 200 based on the sensor measurements. Therefore, the connected shoe 200 may determine whether a correct user or a wrong user is wearing the connected shoe 200, or that the connected shoe 200 is not being worn. At block 730, the determination may be transmitted to a second device, such as the device 100 of FIG. 1, from the connected shoe 200, based on a security policy. The security policy may dictate, e.g., that any change in the status should be immediately transmitted (e.g., as a new determination), and that a reconfirmation should be transmitted when it has been determined that the correct user is wearing the connected shoe 200 and the status has persisted for a predetermined period of time since last transmission. Of course, in some embodiments, sensor measurements may be directly transmitted to the second device, wherein whether a correct user is wearing the connected shoe 200 may be determined at the second device.

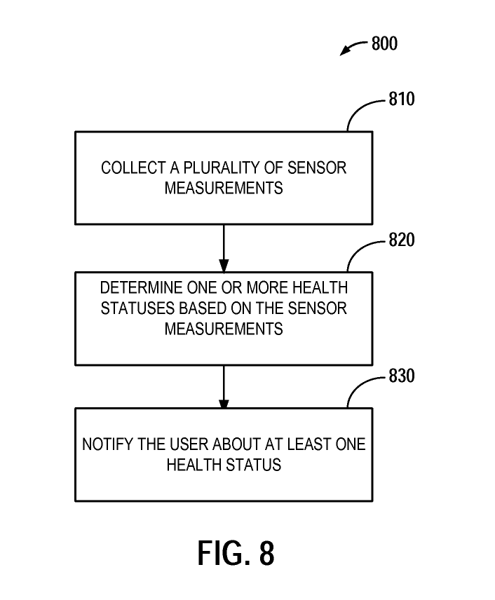

Referring to FIG. 8, a flowchart illustrating an example method 800 for using a connected shoe as a health monitor is shown. At block 810, a plurality of sensor measurements may be collected by a connected shoe 200. These may include, e.g., pressure levels detected using pressure sensor pads 245, one or more motions detected using the motion sensor 250, body temperature levels detected using the body temperature sensor 265, blood pressure levels detected using the blood pressure sensor 260, or user pulse characteristics detected using the pulse sensor 255, etc. At block 820, one or more health statuses may be determined based on the sensor measurements. The health statuses may be determined at the connected shoe 200, or may be determined at a second device, such as the device 100 of FIG. 1, that is wirelessly connected to the connected shoe 200, which first receives the sensor measurements from the connected shoe 200 and then determines the health statuses based on the sensor measurements. The health statuses may include, e.g., a good health inferred from a good gait, a fall (e.g., a major risk for the elderly), a step frequency, a body temperature, a heartrate, or a blood pressure when the user is either at rest or in motion. At block 830, the user may be notified about at least one health status. For example, a message indicating the good health, the step frequency, the body temperature, the heartrate, or the blood pressure levels may be displayed using the second device (if the health statuses are determined at the connected shoe 200, they are first transmitted to the second device before displaying). In another example, one or more vibration motors 230 of the connected shoe 200 may be activated to prompt the user to increase the step frequency in order to reach preset exercise goals, which the user may designate a priori using the second device. The decision to activate the vibration motors 230 may be made at either the second device or the connected shoe 200, based on different embodiments. Some obvious transmissions of data between the second device and the connected shoe 200 have been omitted from the description in order not to obscure the disclosure. In yet another example, when a fall of the user is detected, a status indicating the fall may be transmitted to a preset third party automatically along with other relevant information such as the user's identity and location. The preset third party may include, e.g., an emergency response service, or an emergency contact, etc.

It has also been contemplated that a connected shoe 200 may be wirelessly connected to another wearable device, such as a smartwatch, and as such, the combination of the smartwatch and the connected shoe 200 may provide various functions without the involvement of a conventional smartphone.

Therefore, embodiments of the disclosure are directed to a connected shoe apparatus comprising a processor/controller, a memory coupled to the processor/controller, a battery, radio(s) and antenna(s), or vibration motors, and may further include without limitation piezoelectric components and/or additional sensors such as a magnetometer, a motion sensor, a pulse sensor, a blood pressure sensor, or a body temperature sensor, etc. Various use cases of the connected shoe, depending on different embodiments of the disclosure, have been described herein. These may include without limitation direction detection, navigation with tactile notification, off-loading of power-intensive operations of a wirelessly connected second device, biometric authentication, wearable identity management, or health monitoring, etc. Such feature-rich connected shoes are nevertheless convenient to use as they do not require the user to change any normal, daily habits.

Various implementations of a connected shoe have been previously described in detail. It should be appreciated that application or system that various operations of the connected shoe and of the devices with which the connected shoe is connected may be implemented as software, firmware, hardware, combinations thereof, etc. In one embodiment, the previous described functions may be implemented by one or more processors (e.g., processors 110 of a device 100, processor/controller 220 of a connected shoe 200) to achieve the previously desired functions (e.g., the method operations of FIGS. 4-8). A connected shoe in connection with one or more other devices may provide various useful functions, as have been described in detail above.

Example methods, apparatuses, or articles of manufacture presented herein may be implemented, in whole or in part, for use in or with mobile communication devices. As used herein, “mobile device,” “mobile communication device,” “hand-held device,” “tablets,” etc., or the plural form of such terms may be used interchangeably and may refer to any kind of special purpose computing platform or device that may communicate through wireless transmission or receipt of information over suitable communications networks according to one or more communication protocols, and that may from time to time have a position or location that changes. As a way of illustration, special purpose mobile communication devices, may include, for example, cellular telephones, satellite telephones, smart telephones, heat map or radio map generation tools or devices, observed signal parameter generation tools or devices, personal digital assistants (PDAs), laptop computers, personal entertainment systems, e-book readers, tablet personal computers (PC), personal audio or video devices, personal navigation units, wearable devices, or the like. It should be appreciated, however, that these are merely illustrative examples relating to mobile devices that may be utilized to facilitate or support one or more processes or operations described herein.

The methodologies described herein may be implemented in different ways and with different configurations depending upon the particular application. For example, such methodologies may be implemented in hardware, firmware, and/or combinations thereof, along with software. In a hardware implementation, for example, a processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other devices units designed to perform the functions described herein, and/or combinations thereof.

The herein described storage media may comprise primary, secondary, and/or tertiary storage media. Primary storage media may include memory such as random access memory and/or read-only memory, for example. Secondary storage media may include mass storage such as a magnetic or solid-state hard drive. Tertiary storage media may include removable storage media such as a magnetic or optical disk, a magnetic tape, a solid-state storage device, etc. In certain implementations, the storage media or portions thereof may be operatively receptive of, or otherwise configurable to couple to, other components of a computing platform, such as a processor.

In at least some implementations, one or more portions of the herein described storage media may store signals representative of data and/or information as expressed by a particular state of the storage media. For example, an electronic signal representative of data and/or information may be “stored” in a portion of the storage media (e.g., memory) by affecting or changing the state of such portions of the storage media to represent data and/or information as binary information (e.g., ones and zeros). As such, in a particular implementation, such a change of state of the portion of the storage media to store a signal representative of data and/or information constitutes a transformation of storage media to a different state or thing.

In the preceding detailed description, numerous specific details have been set forth to provide a thorough understanding of claimed subject matter. However, it will be understood by those skilled in the art that claimed subject matter may be practiced without these specific details. In other instances, methods and apparatuses that would be known by one of ordinary skill have not been described in detail so as not to obscure claimed subject matter.

Some portions of the preceding detailed description have been presented in terms of algorithms or symbolic representations of operations on binary digital electronic signals stored within a memory of a specific apparatus or special purpose computing device or platform. In the context of this particular specification, the term specific apparatus or the like includes a general purpose computer once it is programmed to perform particular functions pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated as electronic signals representing information. It has proven convenient at times, principally for reasons of common usage, to refer to such signals as bits, data, values, elements, symbols, characters, terms, numbers, numerals, information, or the like. It should be understood, however, that all of these or similar terms are to be associated with appropriate physical quantities and are merely convenient labels.

Unless specifically stated otherwise, as apparent from the following discussion, it is appreciated that throughout this specification discussions utilizing terms such as “processing,” “computing,” “calculating,” “identifying”, “determining”, “establishing”, “obtaining”, and/or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device. In the context of this particular patent application, the term “specific apparatus” may include a general-purpose computer once it is programmed to perform particular functions pursuant to instructions from program software.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in some implementations” or other like phrases in various places throughout this specification are not necessarily all referring to the same feature, example, and/or limitation. Furthermore, the particular features, structures, or characteristics may be combined in one or more examples and/or features.

While there has been illustrated and described what are presently considered to be example features, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all aspects falling within the scope of appended claims, and equivalents thereof.

What is claimed is:

1. A connected shoe apparatus, comprising:
a processor;
a memory coupled to the processor;
a radio;
an antenna; and
a magnetometer,
wherein the connected shoe apparatus is wearable as a shoe by a user, and wherein the processor is to:
determine a direction the connected shoe apparatus is facing with a magnetometer, and
transmit information associated with the direction to a second device via the radio and the antenna.

2. The connected shoe apparatus of claim 1, further comprising one or more vibration motors, wherein the processor is further to:
receive navigation information from the second device via the radio and the antenna, and
provide tactile navigation notification to the user using vibration motors based on the received navigation information.

3. The connected shoe apparatus of claim 2, wherein a navigation destination is preconfigured with the second device.

4. The connected shoe apparatus of claim 1, further comprising one or more piezoelectric components connected to a battery, the piezoelectric components harvesting energy from the user's walking and recharging the battery, wherein the connected shoe apparatus is connected to the second device via a low energy wireless connection and off-loads power-intensive operations of the second device.

5. The connected shoe apparatus of claim 4, wherein the power-intensive operations off-loaded comprise radio operations or data processing operations.

6. The connected shoe apparatus of claim 1, further comprising at least one of one or more vibration motors, pressure sensor pads, a motion sensor, a pulse sensor, a blood pressure sensor, or a body temperature sensor.

7. The connected shoe apparatus of claim 6, wherein the processor is further to:
receive sensor readings from the pressure sensor pads or the motion sensor,
determine user gait information based on the sensor readings, and
transmit the user gait information to the second device.

8. The connected shoe apparatus of claim 7, wherein the user gait information is used at the second device for user identification or authentication.

9. The connected shoe apparatus of claim 7, wherein the processor is further to:
collect a plurality of sensor measurements,
determine whether a correct user is wearing the connected shoe apparatus based on the sensor measurements, and
transmit the determination to the second device.

10. The connected shoe apparatus of claim 9, wherein the transmission is based on a security policy, and wherein a new determination is transmitted to the second device when the determination changes, and a reconfirmation is transmitted to the second device when a determination that the correct user is wearing the connected shoe apparatus has persisted for a predetermined period of time since last transmission.

11. The connected shoe apparatus of claim 7, wherein the processor is further to:
collect a plurality of sensor measurements,
determine one or more health statuses based on the sensor measurements, and
notify the user about at least one health status.

12. The connected shoe apparatus of claim 11, wherein the health statuses comprise at least one of a good health inferred from a good gait, a fall, a step frequency, a body temperature, a heartrate, or a blood pressure when the user is either at rest or in motion.

13. The connected shoe apparatus of claim 11, wherein the processor is further to in response to a health status indicating a fall, transmit the health status indicating the fall to a preconfigured third party.

14. The connected shoe apparatus of claim 11, wherein the processor is further to transmit at least one health status to the second device for display.

15. The connected shoe apparatus of claim 11, wherein the processor is further to activate one or more vibration motors to prompt the user to increase a step frequency in order to reach preset exercise goals.

16. The connected shoe apparatus of claim 1, wherein the connected shoe apparatus is wirelessly connected to a smartwatch via the radio and the antenna.

17. A method, comprising:
determining a direction a connected shoe apparatus is facing with a magnetometer; and
transmitting information associated with the direction to a second device via a radio and an antenna,
wherein the connected shoe apparatus is wearable as a shoe by a user.

18. The method of claim 17, further comprising:
receiving navigation information from the second device via the radio and the antenna; and
providing tactile navigation notification to the user using vibration motors based on the received navigation information.

19. The method of claim 18, wherein a navigation destination is preconfigured with the second device.

20. The method of claim 17, further comprising off-loading power-intensive operations of the second device,
wherein the connected shoe comprises one or more piezoelectric components connected to a battery, the piezoelectric components harvesting energy from the user's walking and recharging the battery, and wherein the connected shoe is connected to the second device via a low energy wireless connection.

21. The method of claim 20, wherein the power-intensive operations off-loaded comprise radio operations or data processing operations.

22. The method of claim 17, wherein the connected shoe comprises at least one of one or more vibration motors, pressure sensor pads, a motion sensor, a pulse sensor, a blood pressure sensor, or a body temperature sensor.

23. The method of claim 22, further comprising:
receiving sensor readings from the pressure sensor pads or the motion sensor;
determining user gait information based on the sensor readings; and
transmitting the user gait information to the second device.

24. The method of claim 23, wherein the user gait information is used at the second device for user identification or authentication.

25. The method of claim 23, further comprising:
collecting a plurality of sensor measurements;
determining whether a correct user is wearing the connected shoe based on the sensor measurements; and
transmitting the determination to the second device.

26. The method of claim 25, wherein the transmission is based on a security policy, and wherein a new determination is transmitted to the second device when the determination changes, and a reconfirmation is transmitted to the second device when a determination that the correct user is wearing the connected shoe has persisted for a predetermined period of time since last transmission.

27. The method of claim 23, further comprising:
collecting a plurality of sensor measurements;
determining one or more health statuses based on the sensor measurements; and
notifying the user about at least one health status.

28. The method of claim 27, wherein the health statuses comprise at least one of a good health inferred from a good gait, a fall, a step frequency, a body temperature, a heartrate, or a blood pressure when the user is either at rest or in motion.

29. The method of claim 27, further comprising:
in response to a health status indicating a fall, transmitting the health status indicating the fall to a preconfigured third party.

30. The method of claim 27, further comprising transmitting at least one health status to the second device for display.

31. The method of claim 27, further comprising activating one or more vibration motors to prompt the user to increase a step frequency in order to reach preset exercise goals.

32. The method of claim 17, wherein the connected shoe is wirelessly connected to a smartwatch via the radio and the antenna.

33. A connected shoe apparatus, comprising:
means for determining a direction the connected shoe apparatus is facing; and
means for transmitting information associated with the direction to a second device,
wherein the connected shoe apparatus is wearable as a shoe by a user.

34. The connected shoe apparatus of claim 33, further comprising:
means for receiving navigation information from the second device; and
means for providing tactile navigation notification to the user based on the received navigation information.

35. The connected shoe apparatus of claim 34, wherein a navigation destination is preconfigured with the second device.

36. The connected shoe apparatus of claim 33, further comprising means for off-loading power-intensive operations of the second device,
wherein the connected shoe apparatus comprises means for harvesting energy from the user's walking and recharging a battery, and wherein the connected shoe apparatus is connected to the second device via a low energy wireless connection.

37. The connected shoe apparatus of claim 36, wherein the power-intensive operations off-loaded comprise radio operations or data processing operations.

38. The connected shoe apparatus of claim 33, further comprising at least one of one or more vibration means, pressure detection means, a motion detection means, a pulse detection means, a blood pressure detection means, or a body temperature detection means.

39. The connected shoe apparatus of claim 38, further comprising:
means for receiving sensor readings from the pressure detection means or the motion detection means;
means for determining user gait information based on readings from the detection means; and
means for transmitting the user gait information to the second device.

40. The connected shoe apparatus of claim 39, wherein the user gait information is used at the second device for user identification or authentication.

41. The connected shoe apparatus of claim 39, further comprising:
means for collecting a plurality of detection means measurements;
means for determining whether a correct user is wearing the connected shoe apparatus based on the detection means measurements; and
means for transmitting the determination to the second device.

42. The connected shoe apparatus of claim 41, wherein the transmission is based on a security policy, and wherein a new determination is transmitted to the second device when the determination changes, and a reconfirmation is transmitted to the second device when a determination that the correct user is wearing the connected shoe apparatus has persisted for a predetermined period of time since last transmission.

43. The connected shoe apparatus of claim 39, further comprising:
means for collecting a plurality of detection means measurements;
means for determining one or more health statuses based on the detection means measurements; and
means for notifying the user about at least one health status.

44. The connected shoe apparatus of claim 43, wherein the health statuses comprise at least one of a good health inferred from a good gait, a fall, a step frequency, a body temperature, a heartrate, or a blood pressure when the user is either at rest or in motion.

45. The connected shoe apparatus of claim 43, further comprising:
in response to a health status indicating a fall, means for transmitting the health status indicating the fall to a preconfigured third party.

46. The connected shoe apparatus of claim 43, further comprising means for transmitting at least one health status to the second device for display.

47. The connected shoe apparatus of claim 43, further comprising means for activating one or more vibration means to prompt the user to increase a step frequency in order to reach preset exercise goals.

48. The connected shoe apparatus of claim 33, wherein the connected shoe apparatus is wirelessly connected to a smartwatch.

49. A non-transitory computer-readable medium comprising code which, when executed by a processor of a connected shoe wearable by a user, causes the processor to perform a method comprising:
determining a direction the connected shoe is facing with a magnetometer; and
transmitting information associated with the direction to a second device via a radio and an antenna.

50. The non-transitory computer-readable medium of claim 49, further comprising code for:
receiving navigation information from the second device via the radio and the antenna; and
providing tactile navigation notification to the user using vibration motors based on the received navigation information.

51. The non-transitory computer-readable medium of claim 50, wherein a navigation destination is preconfigured with the second device.

52. The non-transitory computer-readable medium of claim 49, further comprising code for off-loading power-intensive operations of the second device,
wherein the connected shoe comprises one or more piezoelectric components connected to a battery, the piezoelectric components harvesting energy from the user's walking and recharging the battery, and wherein the connected shoe is connected to the second device via a low energy wireless connection.

53. The non-transitory computer-readable medium of claim 52, wherein the power-intensive operations off-loaded comprise radio operations or data processing operations.

54. The non-transitory computer-readable medium of claim 49, wherein the connected shoe comprises at least one of one or more vibration motors, pressure sensor pads, a motion sensor, a pulse sensor, a blood pressure sensor, or a body temperature sensor.

55. The non-transitory computer-readable medium of claim 54, further comprising code for:
receiving sensor readings from the pressure sensor pads or the motion sensor;
determining user gait information based on the sensor readings; and
transmitting the user gait information to the second device.

56. The non-transitory computer-readable medium of claim 55, wherein the user gait information is used at the second device for user identification or authentication.

57. The non-transitory computer-readable medium of claim 55, further comprising code for:
collecting a plurality of sensor measurements;
determining whether a correct user is wearing the connected shoe based on the sensor measurements; and
transmitting the determination to the second device.

58. The non-transitory computer-readable medium of claim 57, wherein the transmission is based on a security policy, and wherein a new determination is transmitted to the second device when the determination changes, and a reconfirmation is transmitted to the second device when a determination that the correct user is wearing the connected shoe has persisted for a predetermined period of time since last transmission.

59. The non-transitory computer-readable medium of claim 55, further comprising code for:
collecting a plurality of sensor measurements;
determining one or more health statuses based on the sensor measurements; and
notifying the user about at least one health status.

60. The non-transitory computer-readable medium of claim 59, wherein the health statuses comprise at least one of a good health inferred from a good gait, a fall, a step frequency, a body temperature, a heartrate, or a blood pressure when the user is either at rest or in motion.

61. The non-transitory computer-readable medium of claim 59, further comprising code for:
in response to a health status indicating a fall, transmitting the health status indicating the fall to a preconfigured third party.

62. The non-transitory computer-readable medium of claim 59, further comprising code for transmitting at least one health status to the second device for display.

63. The non-transitory computer-readable medium of claim 59, further comprising code for activating one or more vibration motors to prompt the user to increase a step frequency in order to reach preset exercise goals.

64. The non-transitory computer-readable medium of claim 49, wherein the connected shoe is wirelessly connected to a smartwatch via the radio and the antenna.

65. An article of footwear, comprising:
a processor;
a memory coupled to the processor;
a radio;
an antenna; and
a magnetometer,
wherein the article of footwear is wearable as a footwear by a user, and wherein the processor is to:
determine a direction the article of footwear is facing with a magnetometer, and
transmit information associated with the direction to a second device via the radio and the antenna.

66. The article of footwear of claim 65, further comprising one or more vibration motors, wherein the processor is further to:
receive navigation information from the second device via the radio and the antenna, and
provide tactile navigation notification to the user using vibration motors based on the received navigation information.

67. The article of footwear of claim 66, wherein a navigation destination is preconfigured with the second device.

68. The article of footwear of claim 65, further comprising one or more piezoelectric components connected to a battery, the piezoelectric components harvesting energy from the user's walking and recharging the battery, wherein the article of footwear is connected to the second device via a low energy wireless connection and off-loads power-intensive operations of the second device.

69. The article of footwear of claim 68, wherein the power-intensive operations off-loaded comprise radio operations or data processing operations.

70. The article of footwear of claim 65, further comprising at least one of one or more vibration motors, pressure sensor pads, a motion sensor, a pulse sensor, a blood pressure sensor, or a body temperature sensor.

71. The article of footwear of claim 70, wherein the processor is further to:
receive sensor readings from the pressure sensor pads or the motion sensor,
determine user gait information based on the sensor readings, and
transmit the user gait information to the second device.

72. The article of footwear of claim 71, wherein the user gait information is used at the second device for user identification or authentication.

73. The article of footwear of claim 71, wherein the processor is further to:
collect a plurality of sensor measurements,
determine whether a correct user is wearing the article of footwear based on the sensor measurements, and
transmit the determination to the second device.

74. The article of footwear of claim 73, wherein the transmission is based on a security policy, and wherein a new determination is transmitted to the second device when the determination changes, and a reconfirmation is transmitted to the second device when a determination that the correct user is wearing the article of footwear has persisted for a predetermined period of time since last transmission.

75. The article of footwear of claim 71, wherein the processor is further to:
collect a plurality of sensor measurements,
determine one or more health statuses based on the sensor measurements, and
notify the user about at least one health status.

76. The article of footwear of claim 75, wherein the health statuses comprise at least one of a good health inferred from a good gait, a fall, a step frequency, a body temperature, a heartrate, or a blood pressure when the user is either at rest or in motion.

77. The article of footwear of claim 75, wherein the processor is further to in response to a health status indicating a fall, transmit the health status indicating the fall to a preconfigured third party.

78. The article of footwear of claim 75, wherein the processor is further to transmit at least one health status to the second device for display.

79. The article of footwear of claim 75, wherein the processor is further to activate one or more vibration motors to prompt the user to increase a step frequency in order to reach preset exercise goals.

80. The article of footwear of claim 65, wherein the article of footwear is wirelessly connected to a smartwatch via the radio and the antenna.
